(19) United States
(12) Patent Application Publication
(10) Pub. No.: US 2008/0194614 A1
(43) Pub. Date: Aug. 14, 2008

(54) Combination of Organic Compounds

(76) Inventors: Richard Jean Dorent, Eaubonne (FR); Carole Anne Sips, Pfastaff (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/915,623

(22) PCT Filed: May 29, 2006
(86) PCT No.: PCT/EP2006/005108

§ 371 Date: Nov. 27, 2007

Related U.S. Application Data
(60) Provisional application No. 60/685,849, filed on May 31, 2005.

(51) Int. Cl. A61K 31/4353; A61K 31/404; A61P 9/00; A61P 3/00; A61P 1/00; A61P 35/00; A61P 19/02; A61K 31/47

(52) U.S. Cl. 514/291; 514/415; 514/311

(57) ABSTRACT

The invention relates to a pharmaceutical combination comprising an HMG-Co-A reductase inhibitor, especially fluvastatinor pitavastatin or a pharmaceutically acceptable salt thereof and mTOR inhibiting agent, e.g. rapamycin or a rapamycin derivative.

The present invention relates to a combination, such as a combined preparation or pharmaceutical composition, respectively, comprising an HMG-Co-A reductase inhibitor (also called β-hydroxy-β-methylglutaryl-co-enzyme-A reductase inhibitor) or pharmaceutically acceptable salts thereof and a mTOR inhibiting agent, e.g. rapamycin or a rapamycin derivative, optionally in the presence of a pharmaceutically acceptable carrier for simultaneous, separate or sequential use, especially in the prevention, delay of progression or treatment of HMG-Co-A reductase inhibitors related conditions or diseases such as hypercholesterolemia, mixed dyslipidemia, secondary prevention of cardiovascular event, atherosclerosis and in the in the prevention, delay of progression or treatment of mTOR inhibiting agent related conditions or diseases, the use of such combination for the preparation of a pharmaceutical preparation for the prevention, delay of progression or treatment of such conditions; a method of prevention, delay of progression or treatment of HMG-Co-A reductase inhibitors related conditions or diseases such as hypercholesterolemia, mixed dyslipidemia, secondary prevention of cardiovascular event, atherosclerosis and mTOR inhibiting agent related conditions or diseases such as transplanumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption.

The present invention relates to pharmaceutical combinations or compositions comprising an HMG-Co-A reductase inhibitor or pharmaceutically acceptable salts thereof and a mTOR inhibiting agent, e.g. rapamycin or a rapamycin derivative, optionally in the presence of a pharmaceutically acceptable carrier and their uses in treating HMG-Co-A reductase inhibitors related conditions or diseases such as hypercholesterolemia, mixed dyslipidemia, secondary prevention of cardiovascular event, atherosclerosis like hypercholesterolemia and mTOR inhibiting agent related conditions or diseases such as transplantation, Rheumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption.

The present invention furthermore relates to pharmaceutical combinations or compositions which comprise in combination an HMG-Co-A reductase inhibitor selected from the list of atorvastatin, cerivastatin, fluvastatin, lovastatin, pitavastatin (formerly itavastatin), pravastatin, rosuvastatin, and simvastatin, or, in each case, a pharmaceutically acceptable salt thereof, (preferred is fluvastatin, atorvastatin, pitavastatin or simvastatin or a pharmaceutically acceptable salt thereof) and a mTOR inhibiting agent, e.g. rapamycin or a rapamycin derivative and optionally a pharmaceutically acceptable carrier for simultaneous, sequential or separate use.

In a preferred embodiment, the present invention relates to pharmaceutical combinations or compositions which comprise in combination fluvastatin or pitavastatin or a pharmaceutically acceptable salt thereof and a mTOR inhibiting agent, e.g. rapamycin or a rapamycin derivative optionally a pharmaceutically acceptable carrier for simultaneous, sequential or separate use.

In another preferred embodiment, the present invention relates to pharmaceutical combinations or compositions which comprise in combination fluvastatin or pitavastatin or a pharmaceutically acceptable salt thereof and a mTOR inhibiting agent, e.g. rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 40-0-(2-hydroxyethyl)-rapamycin, 32-deoxorapamycin and 16-pent-2-ynyloxy-32(S)-dihydro-rapamycin and optionally a pharmaceutically acceptable carrier for simultaneous, sequential or separate use.

In another preferred embodiment, the present invention relates to pharmaceutical combinations or compositions which comprise in combination fluvastatin or pitavastatin or a pharmaceutically acceptable salt thereof and 40-0-(2-hydroxyethyl)-rapamycin and optionally a pharmaceutically acceptable carrier for simultaneous, sequential or separate use.

In one aspect the present invention relates to pharmaceutical combinations or compositions according to the invention for the treatment or prevention of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases which comprise in combination an HMG-Co-A reductase inhibitor, especially pitavastatin or fluvastatin or a pharmaceutically acceptable salt thereof and a mTOR inhibiting agent, e.g. rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 40-0-(2-hydroxyethyl)-rapamycin, 32-deoxorapamycin and 16-pent-2-ynyloxy-32(S)-dihydro-rapamycin and optionally a pharmaceutically acceptable carrier for simultaneous, sequential or separate use.

In one aspect the present invention relates to pharmaceutical combinations or compositions according to the invention for the treatment or prevention of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases which comprise in combination fluvastatin or pitavastatin or a pharmaceutically acceptable salt thereof and the mTOR inhibiting agent 40-0-(2-hydroxyethyl)-rapamycin and optionally a pharmaceutically acceptable carrier for simultaneous, sequential or separate use.

In a preferred aspect the present invention relates to pharmaceutical combinations or compositions according to the invention for the treatment or prevention of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases which comprise in combination fluvastatin or pitavastatin or a pharmaceutically acceptable salt thereof and the mTOR inhibiting agent 40-0-(2-hydroxyethyl)-rapamycin and optionally a pharmaceutically acceptable carrier for simultaneous, sequential or separate use.

In another aspect the present invention relates to pharmaceutical combinations or compositions according to the invention for the treatment or prevention of mTOR inhibiting agent related conditions or diseases such as transplantation, Rheumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption, which comprise in combination (i) fluvastatin, atorvastatin, pitavastatin or simvastatin or a pharmaceutically acceptable salt thereof and (ii) a mTOR inhibiting agent, e.g. rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 40-0-(2-hydroxyethyl)-rapamycin, 32-deoxorapamycin and 16-pent-2-ynyloxy-32(S)-dihydro-rapamycin and optionally a pharmaceutically acceptable carrier for simultaneous, sequential or separate use.

In a preferred aspect the present invention relates to pharmaceutical combinations or compositions according to the invention for the treatment or prevention of mTOR inhibiting agent related conditions or diseases such as transplanumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption, which comprise in combination (i) fluvastatin or pitavastatin or a pharmaceutically acceptable salt thereof and (ii) the mTOR inhibiting agent and 40-0-(2-hydroxyethyl) and optionally a pharmaceutically acceptable carrier for simultaneous, sequential or separate use.

In these compositions, components (i) and (ii) can be obtained and administered together, one after the other or separately in one combined unit dose form or in two separate unit dose forms. The unit dose form may also be a fixed combination.

In another embodiment, the invention provides the use of a pharmaceutical combination according to the invention for the preparation of a medicament for the treatment or prevention HMG-Co-A reductase inhibitors related conditions or diseases such as hypercholesterolemia, mixed dyslipidemia, secondary prevention of cardiovascular event, atherosclerosis and mTOR inhibiting agent related conditions or diseases such as transplanumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption.

In another embodiment, the invention provides the use of a pharmaceutical composition according to the invention for the treatment or prevention of HMG-Co-A reductase inhibitors related conditions or diseases such as hypercholesterolemia related conditions or diseases, mixed dyslipidemia related conditions or diseases, secondary prevention of cardiovascular event and atherosclerosis related conditions or diseases.

In another embodiment, the invention provides the use of a pharmaceutical composition according to the invention for the treatment or prevention and mTOR inhibiting agent related conditions or diseases such as transplanumatoid arthritis, Inflammatory Bowel Disease (IBD),

chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption.

The present invention provides a kit comprising in separate containers in a single package pharmaceutical combinations or compositions comprising in one container a pharmaceutical composition comprising an HMG-Co-A reductase inhibitor, especially pitavastatin or fluvastatin or a pharmaceutically acceptable salt thereof, and in a second container a pharmaceutical composition comprising a mTOR inhibiting agent, e.g. rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 40-0-(2-hydroxyethyl)-rapamycin, 32-deoxorapamycin and 16-pent-2-ynyloxy-32(S)-dihydro-rapamycin.

The present invention provides a kit comprising in separate containers in a single package pharmaceutical combinations or compositions comprising in one container a pharmaceutical composition comprising fluvastatin or pitavastatin, and in a second container the mTOR inhibiting agent 40-0-(2-hydroxyethyl)-rapamycin.

The kit form is particularly advantageous when the separate components must be administered in different dosage forms or are administered at different dosage intervals.

The present invention relates to a package comprising an HMG-Co-A reductase inhibitor, especially fluvastatin or pitavastatin together with instructions for use in combination with a mTOR inhibiting agent, e.g. rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 40-0-(2-hydroxyethyl)-rapamycin, 32-deoxorapamycin and 16-pent-2-ynyloxy-32(S)-dihydro-rapamycin for the treatment or prevention of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases and mTOR inhibiting agent related conditions or diseases such as transplantation, Rheumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption.

In a preferred embodiment, the package according to the invention comprises in combination fluvastatin or pitavastatin or a pharmaceutically acceptable salt thereof and the mTOR inhibiting agent 40-0-(2-hydroxyethyl)-rapamycin.

In another embodiment the present invention relates to methods of prevention or treatment of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases comprising the administration of a therapeutically effective amount of any preferred pharmaceutical composition according to the invention and optionally a pharmaceutically acceptable carrier to a mammal in need thereof.

In another embodiment the present invention relates to methods of prevention or treatment of mTOR inhibiting agent related conditions or diseases such as transplantation, Rheumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption comprising the administration of a therapeutically effective amount of any preferred pharmaceutical composition according to the invention and optionally a pharmaceutically acceptable carrier to a mammal in need thereof.

In a preferred embodiment the present invention relates to methods of prevention or treatment of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases comprising the administration of a therapeutically effective amount of fluvastatin or pitavastatin or a pharmaceutically acceptable salt thereof and the mTOR inhibiting agent 40-0-(2-hydroxyethyl)-rapamycin and optionally a pharmaceutically acceptable carrier to a mammal in need thereof.

In a preferred embodiment the present invention relates to methods of prevention or treatment of mTOR inhibiting agent related conditions or diseases such as transplantation, Rheumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption comprising the administration of a therapeutically effective amount of fluvastatin or pitavastatin or a pharmaceutically acceptable salt thereof and the mTOR inhibiting agent 40-0-(2-hydroxyethyl)-rapamycin and optionally a pharmaceutically acceptable carrier to a mammal in need thereof.

HMG-Co-A reductase inhibitors (also called β-hydroxy-β-methylglutaryl-co-enzyme-A reductase inhibitors) are understood to be those active agents which may be used to lower the lipid levels including cholesterol in blood.

The class of HMG-Co-A reductase inhibitors comprises compounds having differing structural features. For example, mention may be made of the compounds which are selected from the group consisting of atorvastatin, cerivastatin, fluvastatin, lovastatin, pitavastatin (formerly itavastatin), pravastatin, rosuvastatin, and simvastatin, or, in each case, a pharmaceutically acceptable salt thereof.

Preferred HMG-Co-A reductase inhibitors are those agents which have been marketed, most preferred is fluvastatin, atorvastatin, pitavastatin or simvastatin or a pharmaceutically acceptable salt thereof.

Methods of making the HMG-CoA reductase inhibitors are well known by those skilled in the art and such agents include those commercially available.

The HMG-CoA reductase inhibitors may be used in their free acid forms, in their ester forms, or as their pharmaceutically acceptable salts. Such pharmaceutically acceptable salts include, for example, sodium salts, calcium salts, and ester salts.

The HMG-CoA reductase inhibitors may be used as racemic mixtures, or as a more active stereoisomer as appropriate.

The HMG-CoA reductase inhibitors may be present in an amount effective to inhibit biosynthesis of cholesterol in humans. In one embodiment, the pharmaceutical compositions comprise from about 5 to about 50 weight percent of the HMG-CoA reductase inhibitor, based on total weight of the composition. More preferably, the compositions comprise from about 20 to about 40 weight percent of the HMG-CoA reductase inhibitor, based on total weight of the composition.

A mTOR inhibitor is a compound which targets intracellular mTOR (“mammalian Target Of Rapamycin”). mTOR is a family member of phosphatidylinositol 3-kinase (P13-kinase) related kinase. Rapamycin and rapamycin derivatives inhibit the mTOR pathway via a complex with its intracellular receptor FKBP12 (FK506-binding protein 12).

Rapamycin is a known macrolide antibiotic produced by Streptomyces hygroscopicus. By rapamycin derivative is meant a substituted rapamycin having mTOR inhibiting properties, e.g. rapamycin substituted in position 40 and/or 16 and/or 32, for example a compound of formula I

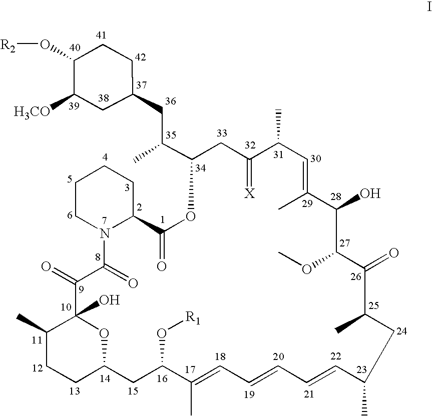

wherein
R₁ is CH₃ or C₃₋₆alkynyl,
R₂ is H, —CH₂—CH₂-OH, 3-hydroxy-2-(hydroxymethyl)-2-methyl-propanoyl or tetrazolyl, and

### X is ═O, (H,H) or (H,OH)

provided that R₂ is other than H when X is ═O and R₁ is CH₃,
or a prodrug thereof when R₂ is —CH₂—CH₂-OH, e.g. a physiologically hydrolysable ether thereof.

Representative rapamycin derivatives of formula I are e.g. 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779) or 40-epi-(tetrazolyl)-rapamycin (also called ABT578). A preferred compound is e.g. 40-0-(2-hydroxyethyl)-rapamycin disclosed in Example 8 in WO 94/09010 (referred hereinafter as Compound A), or 32-deoxorapamycin or 16-pent-2-ynyloxy-32(S)-dihydro-rapamycin as disclosed in WO 96/41807.

Rapamycin derivatives may also include the so-called rapalogs, e.g. as disclosed in WO 98/02441 and WO01/14387, e.g. AP23573, AP23464, AP23675 or AP23841.

Further examples of a rapamycin derivative are those disclosed under the name TAFA-93, biolimus-7 or biolimus-9.

It has surprisingly been found that the pharmaceutical combinations or compositions according to the invention can be used for the treatment or prevention of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases and mTOR inhibiting agent related conditions or diseases such as transplantation, Rheumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption.

It has surprisingly been found that, a combination of an HMG-Co-A reductase inhibitor, especially fluvastatin or pitavastatin or a pharmaceutically acceptable salt thereof, and a mTOR inhibiting agent, e.g. rapamycin or a rapamycin derivative achieves greater therapeutic effect (a potentiation) than the administration of fluvastatin or pitavastatin or the mTOR inhibiting agent, e.g. rapamycin or a rapamycin derivative agent alone.

Preferably the below experimental parts studies carried out with 1) a combination comprising pitavastatin or a pharmaceutically acceptable salt thereof and 40-0-(2-hydroxyethyl)-rapamycin, 2) pitavastatin alone and 3) 40-0-(2-hydroxyethyl)-rapamycin alone.

Preferably the below experimental studies are carried out with 1) a combination comprising fluvastatin or a pharmaceutically acceptable salt thereof and 40-0-(2-hydroxyethyl)-rapamycin, 2) fluvastatin alone and 3) 40-0-(2-hydroxyethyl)-rapamycin alone.

For example, representative studies are carried out with a combination of fluvastatin andeverolimus, e.g, applying the following methodology:

We have evaluated the antiatherotrombotic effect of everolimus (40-0-(2-hydroxyethyl)-rapamycin) alone or in combination with fluvastatin or pitavastatin with the final aim to address the potential additive or synergistic benefits of this combination of drugs with different mechanism of action.

### Rabbit Carotid Artery Injury: an Experimental Model of Atherothrombosis.

In order to investigate the potential pharmacological modulation of atherothrombosis it is fundamental to utilize an experimental model which resembles many of the events occurring during atherogenesis and its thromboembolic complications. We have established an in vivo model of atherogenesis based on the perivascular manipulation of rabbit carotid arteries by surgical insertion of a soft hollow silicone collar (“the collar model”). This approach induces, within two weeks, a reproducible hyperplastic intimal lesion characterized by migration and proliferation of SMCs that arise in the presence of an intact endothelium. In particular, we were able to detect leukocyte (T-lymphocyte, PMN, monocyte) adhesion and infiltration as well as the expression of adhesion molecules such as ICAM-1 and VCAM-1. In this model, by transmission electron microscopy, we have also recently observed a previously unknown interaction between medial polymorphonuclear leukocytes and SMC, referred to as emperipolesis, an active phenomenon of cells engulfing other cells distinct from phagocytosis. The lesion previously described is obtained independently of lipid elevation; however, hypercholesterolemia has a general detrimental effect on the atherogenic processes occurring in this model, leading to a more severe and complicated intimal thickening characterised by abundant ECM, lipid deposition, and cholesterol-loaded monocyte/macrophages. During the last decade, we were able to show the ability of several class of drugs (e.g. statins, calcium antagonists, apoprotein Al-Milano) to inhibit the formation of the plaque occurring after collaring positioning in both normo- and hypercholesterolemic animals, as well as the mechanism of action of the tested compounds. In particular, we demonstrated that fluvastatin or pitavastatin are able to interfere with plaque formation through its primary action (i.e. inhibition of HMG-CoA reductase).

We have also demonstrated an up-regulation of TF (tissue factor) and MMPs (matrix metalloproteinases) expression and increased cholesterol esterification rate in the carotid wall, following perivascular manipulation in hypercholesterolemic rabbits (manuscript in preparation). High TF expression confers a prothrombogenic phenotype to the carotid artery, however, fluvastatin or pitavastatin were shown to attenuate the inflammatory and pro-thrombogenic properties of the atherosclerotic lesions.

### Statins and Atherosclerosis

Clinical trials have firmly established that HMG-CoA reductase inhibitors can induce regression of vascular atherosclerosis as well as reduction of cardiovascular-related morbidity and death in patients with and without coronary artery disease. These beneficial effects on coronary events have generally been attributed to the hypocholesterolemic properties of statins. However, because mevalonate, the product of the enzyme reaction, is the precursor not only of cholesterol but also of many nonsteroidal isoprenoid compounds, inhibition of HMG-CoA reductase may result in pleiotropic effects. Indeed, the mevalonate pathway yields a series of isoprenoids that are vital for diverse cellular functions. Several proteins post-translationally modified by the covalent attachment of mevalonate-derived isoprenoid groups, either farnesyl- or geranylgeranyl-pyrophosphate, have been identified. These proteins must be prenylated as a prerequisite for membrane association, which is required for their function. Members of this family are involved in a number of cellular processes including cell signaling, cell differentiation and proliferation, myelination, cytoskeleton dynamics and endocytotic/exocytotic transport.

A variety of experimental data, indicate that statins, through the inhibition of HMG-CoA reductase, could affect several processes involved in the formation of atherosclerotic lesions, independently of their hypocholesterolemic properties.

The beneficial effect of statins on clinical events may involve non-lipid-related mechanisms that modify endothelial function, inflammatory responses, oxidative modification of circulating lipoproteins, foam cell formation, smooth muscle cell activation, angiogenesis, plaque stability and thrombus formation. The pleiotropic profile of statins can probably be explained by the modulation of the mevalonate pathway, because starvation of mevalonate (as a result of the inhibition of HMG-CoA reductase by statins) has consequences for cellular function that extend beyond decreased cholesterol synthesis. The available data demonstrate that HMG-CoA reductase inhibitors, beyond their lipid-lowering properties, exert a direct antiatherosclerotic effect on the arterial wall that could significantly prevent cardiovascular disease.

### Immunosuppressant Agents and Atherosclerosis.

Rapamycin (sirolimus), a macrolide immunosuppressant inhibitor of mTOR (mammalian target of Rapamycin), inhibits growth factor-dependent proliferation of haematopoietic and nonhaematopoietic cells via cell-cycle arrest in the late G1 phase. Sirolimus has been shown to inhibit vascular SMCs (smooth muscle cells) proliferation and migration in vitro and to affect neointimal growth in balloon-injured rat carotid and porcine coronary arteries. More recently, sirolimus-coated stents were shown to inhibit in-stent neointimal growth in porcine coronary arteries at 28 days, and impressive initial results with sirolimus-eluting stents in humans (0% restenosis rate at 210 days) have been reported.

An orally active immunosuppressant and antiproliferative compound of the same family as sirolimus, everolimus [40-O-(2-hydroxyethyl)-rapamycin], has also shown promising effects in preventing rejection in renal and heart transplantation. Everolimus exhibits potent inhibition of growth factor-induced proliferation of lymphocytes, as well as other hematopoietic and nonhematopoietic cells of mesenchymal origin. Similarly to sirolimus, the biologic activity of everolimus depends on its binding to the immunophyllin—FK506 binding protein 12 (FKBP12). Everolimus-FKBP12 complex interacts with mTOR, a tyrosine kinase essential for progression of the cell cycle from G1 to S phase, later identified as FRAP kinase.

Everolimus prolongs allograft survival in several experimental animal transplant models, and newer data suggest that it may be beneficial in preventing the vasculopathy associated with chronic allograft dysfunction. The experience using everolimus in cardiac transplantation has also provided potentially important insights into the consequences of antiproliferative effects on vascular SMC and fibroblasts where reduction of intimal expansion was identified by intravascular coronary ultrasound examination among those patients receiving everolimus. Such effect presents additional therapeutic targets of potential relevance.

In a rabbit model, oral everolimus at dosages similar to those used for immunosuppression prevented stent-associated neointimal expansion. Perhaps most promising for transplantation were results obtained using everolimus for immunosuppression in first-time recipients of cardiac transplants where a dramatic reduction of the incidence of allograft coronary arteriosclerosis was observed.

### In Vivo Study

Four groups of animals (10 animals per group) are used:
no treatment (control)
everolimus (proposed dosage in the range 0.75-1.5 mg/kg/day)
fluvastatin or pitavastatin (proposed dosage 5 mg/kg/day)
everolimus (proposed dosage in the range 0.75-1.5 mg/kg/day) plus fluvastatin or pitavastatin (proposed dosage 5 mg/kg/day).

On these animals we measure:
plasma lipid profile (total cholesterol, HDL-cholesterol, triglycerides);
lipid accumulation in the carotid artery;
cellular processes involved in lesion formation, namely SMCs accumulation, and leukocyte (particularly monocytes/macrophages, PMNs, T-lymphocytes) infiltration;
expression of adhesion molecules (VCAM-1, ICAM-1, and α1 integrin) on endothelial cells and SMCs;
macrophage functions critical to lesion complication and plaque stability, namely MMPs expression and activity;
expression of Tissue Factor in arterial lesions;
collagen deposition and remodelling;

### In Vitro Study

To gain further insight into the molecular mechanism of the antiatherothrombotic effect of everolimus alone or in combination with fluvastatin or pitavastatin, cultured rabbit and rat vascular arterial SMCs, and mouse peritoneal macrophages are utilized. In addition, human skin fibroblasts (HSF) and the human hepatoma cell line Hep-G2, representing peripheral and central model of lipoprotein metabolism, are utilized with the final aim to evaluate the effect of everolimus on lipoprotein metabolism.

In particular, we will evaluate the effect of the tested drugs on:
SMC proliferation. This in vitro studies allow us to evaluate the potential additive or synergistic effect of the combination of everolimus +/−fluvastatin or pitavastatin. Isobologram analysis will be performed to address this issue as described previously⁵²
Cellular lipoprotein metabolism. This in vitro approach is very useful for investigating possible mechanism(s) responsible for the hyperlipidemic effect of everolimus. More specifically, we explore the effect of everolimus on lipoprotein catabolism and cholesterol metabolism in HSF and in Hep-G2
Cellular cholesterol homeostasis. We study cholesterol synthesis, esterification and efflux to have a clear picture on the effect of everolimus, alone or in combination with fluvastatin or pitavastatin, on lipid metabolism, homeostasis and deposition in vascular cells.
MMP expression and activity. This investigation is very informative to explain some of the potential anti-atherosclerotic effects of everolimus alone or in combination with fluvastatin or pitavastatin.

### Materials and Methods

### In Vivo Studies

Experimental design—The effect of tested drugs on collar-induced carotid lesion is evaluated in hypercholesterolemic rabbits 14 days after collar positioning. Rabbits receive the drugs either by oral gavage (everolimus) or mixed with the diet (fluvastatin or pitavastatin) starting on the day of perivascular manipulation. Hypercholesterolemia is induced by the administration of a cholesterol-rich diet (1% cholesterol) starting 4 weeks before collar insertion.

Plasma lipid evaluation—Blood samples is drawn after overnight fasting from the ear central artery at baseline, at surgery, and at sacrifice to perform lipid analysis. Total-, HDL-cholesterol, and triglycerides levels is determined enzymatically. Overall changes in lipid is calculated by the Area Under the Curve (AUC) of lipid concentration vs. time, using the trapezoidal rule.

ACAT activity (activity acyl-coenzyme cholesterol acyl transferase)) and cholesterol content in carotid—ACAT activity is determined essentially by the method of Helgerud. Carotid rings is homogenised in TRIS/sucrose buffer containing [¹⁴C]-oleoyl coenzyme A (0.5 μCi/sample) complexed with bovine serum, in 0.1 M potassium phosphate buffer, ph 7.4. After incubation for 2 h at 37° C., the reaction is stopped by the addition of 5 ml of chloroform/methanol (2:1 v/v), and lipids extracted. After centrifugation, the chloroform layer is dried under N₂ flux. For the determination of cholesterol content in aortic arch, the same procedure is followed, but omitting the addition of [¹⁴C]-oleoyl coenzyme A in the reaction mixture. The extracted lipids is separated by thin layer chromatography (t.l.c.) (isooctane/diethyl ether/acetic acid, 75:25:2, v/v/v). Cholesterol radioactivity in the spots is determined by liquid scintillation counting (Insta-Fluor, Packard, Groningen, The Netherlands) while cholesterol mass content in the spots is determined by an enzymatic method. We tested the linearity of this method between 1.5 and 50 μg of cholesterol (r²=0.99). In every determination, [³H]-cholesterol was added as internal standard with a recovery of more than 90%.

Carotid lesion—Male New Zealand White rabbits (2.7-3.0 kg) is anaesthetized by intramuscular injection of xylazine (5 mg/kg) and ketamine (35 mg/kg). Animals are then placed in dorsal recumbence and a midline neck incision is made to surgically expose both carotid arteries. A nonocclusive, biologically inert, soft, hollow Silastic collar (SILICOLLAR®, MediGene Oy, Kuopio, Finland) is positioned around both carotid arteries. The collar is 25 mm long and it touches the artery circumference at two points, 20 mm apart. In each animal, the controlateral carotid artery is sham-operated by placing the collar around the artery but removing it just before wounds suturing. At the end of the study, animals are killed by administration of a lethal i.v. dose of urethane (10 ml, 25% aqueous solution) and samples from the carotid arteries are collected and processed according to the appropriate procedures for the different methods of analysis.

Histology—Segments from the carotid arteries are readily dissected and excised just after euthanasia. The arteries are frozen or paraffin embedded, and transversally cut in order to obtain 5 □m serial sections. Tissues are stained with hematoxylin and eosin to identify and quantify vascular structures by morphometric analysis. The following parameters are measured by computer-assisted image analysis (OPTIMAS 6.2, Media Cybernetics, Silver Spring, Md., USA): lumen area (L), area surrounded by the internal elastic lamina (IEL), and area surrounded by the external elastic lamina (EEL). The following parameters are then determined: (a) intimal area=I=IEL−L; (b) medial area=M=EEL−IEL; and (c) intima to media ratio=I/M. Additional sections are stained with picrosirius red dye to label collagen. Picrosirius red positive regions within the lesion are measured using computer-assisted color image analysis.

Immunohistochemical detection of the expression of adhesion molecules (VCAM-1, ICAM-1 and α1 integrin) on endothelial cells and SMC—Identification of the cell adhesion molecules in the intimal carotid lesion is performed using antibodies to ICAM-1, VCAM-1 (R&D system), and α1 integrin (Chemicon). According to standard procedures carotid criosections are incubated with the specific primary antibody and then with a biotinylated species-specific secondary antibody (Vector Laboratories Inc., Burlingame, Calif., USA). Labelling is done with an avidin-biotin-peroxidase kit (Vectastain ABC Elite, Vector Laboratories Inc.) followed by 3,3-diaminobenzidine (Sigma). For negative control the primary antibody is omitted and sections will be incubated with normal horse serum.

VCAM-1, ICAM-1 and α1 integrin positive regions within the lesion is measured using computer-assisted color image analysis.

Quantitative analysis of monocyte-derived macrophages and T lymphocytes accumulation within the lesion—Identification of the leukocyte subsets infiltrating the intimal carotid lesion is performed using antibodies to markers of all leukocytes (anti-CD18, Serotec), polimorphonuclear cells (PMNs)(polynuclear neutrophils) (MCA 805, Serotec), monocyte/macrophages (RAM11, DAKO) and T lymphocytes (anti-CD5, Serotec), according to standard procedures: tissue sections will be incubated with the specific primary antibody and then with a biotinylated species-specific secondary antibody (Vector Laboratories Inc.). Labelling is done with an avidin-biotin-peroxidase kit (Vectastain ABC Elite, Vector Laboratories Inc.) followed by 3,3-diaminobenzidine (Sigma). For negative control the primary antibody is omitted and sections will be incubated with normal horse serum. Total leukocyte, PMN, monocyte/macrophage and T lymphocyte area, identified respectively as CD18-, MCA805-, RAM11- and CD5-positive regions within the lesion are measured using computer-assisted colour image analysis.

Immunohistochemical detection of Tissue Factor and quantitative analysis of Tissue Factor protein expression—For immunohistochemical detection of Tissue Factor, predigested tissue sections are incubated with a specific mouse anti-rabbit tissue factor antibody (AP-1) and then with a biotinylated horse anti-mouse IgG secondary antibody (Vector Laboratories Inc.). Labelling is done with avidin-biotin-peroxidase kit (Vectastain ABC Elite, Vector Laboratories Inc.) followed by 3,3-diaminobenzidine (Sigma), according with the standard ABC method (Vector). For negative control the primary antibody is omitted and sections will be incubated with normal horse serum. The extent of Tissue Factor immunopositive intimal areas is measured using computer-assisted color image analysis.

Analysis of MMPs expression and activity—The distribution of different MMPs is evaluated by immunohistochemistry. Sections are incubated with primary monoclonal antibody (Amersham-Pharmacia-Biotech, UK) and then with biotinylated species-specific secondary antibody (Vector Laboratories Inc.,). Labelling will be performed with FITC-conjugated extrAvidin. Immunostaining of serial sections with anti-MMPs antibodies and cell-specific antibodies (anti- [symbol] actin for SMC, anti-CD31 for endothelial cells and anti-CD18 for leukocytes) are performed to identify the predominant cell type(s) responsible for the expression of the different MMPs.

MMP activity is measured in homogenate of rabbit carotid by gelatin gel zymography.

### In Vitro Studies

Cell isolation and cultures—Mouse peritoneal macrophages (MPM) are collected by peritoneal lavage with phosphate buffered saline (PBS) from mice given a 3 ml intraperitoneal injection of 4% thioglycollate in water. The MPM are pelletted, washed twice with serum-free Dulbecco Modified Eagle (DME) medium, and plated at a density of 3×10⁶ cells/35 mm dish, and allowed to adhere to dishes for 2 h in DME medium containing 10% foetal bovine serum (FBS). Then plates are washed three times with DME medium to remove non-adherent cells, and incubated in DME medium containing 10% FBS until the day of the experiment.

Human skin fibroblasts (HSF) are grown from explants of skin biopsies obtained from normolipidemic clinically healthy individuals. Fibroblasts are characterized in terms of receptor-mediated LDL binding, internalization and degradation. Cells are grown in monolayers and maintained in 75 cm² plastic flasks at 37° C. in a humidified atmosphere of 95% air, 5% CO₂ in F-11 medium supplemented with 10% FCS, non essential aminoacid solution (1%, v:v), penicillin (100 U/ml), streptomycin (100 ug/ml), tricine-buffer (20 mM, pH 7.4), NaHCO₃ (24 mM). For all experiments, cells from the stock flasks are dissociated with 0.05% trypsin—0.02% EDTA at confluency (five to fifteen passages), seeded in 35 mm plastic Petri dishes (1-1.5×10⁵ cells for the experiment of binding uptake and degradation are used just before reaching confluency, usually 6 days after plating and the medium is changed every 2-3 days. For the measurement of cholesterol and of fatty acid synthesis cells are seeded in 35 mm plastic Petri dishes (7.5×10⁵ cells) and incubated with MEM supplemented with 10% FCS. Twenty-four hours later the medium is changed to one containing 10% LPDS, and the cultures are incubated for 24 h. At this time (time 0) the medium is replaced by one containing 10% LPDS in the presence or absence of known concentrations of the tested compounds and the incubation is continued for further 72 h at 37° C. Cholesterol synthesis is estimated by measuring the incorporation of [¹⁴C] acetate into cellular sterols²⁹.

The human hepatoma cell line, Hep-G2, representing the central model of lipoprotein metabolism, obtained from the American Type Culture Collection, is grown in monolayers and cultured as described for HSF with the addition to the medium of 0.11 g/l sodium pyruvate. For all experiments cells are seeded in 35 mm dishes (3-5×10 5 cells) in 2 ml of medium containing 10% FCS and used 6 days after plating.

SMCs are isolated from intima-media layers of aortae of male Sprague Dawley rats or of carotids of male New Zealand White rabbits. Cells are grown in MEM supplemented with 10% (v/v) fetal calf serum (FCS), 100 U/ml penicillin, 0.1 mg/ml streptomycin, 20 mM tricine buffer and 1% (v/v) non-essential amino acid solution. Cells are used between the 4th and 10th passage. SMCs are identified for growth behaviour, morphology and using a monoclonal antibody specific for α-actin (Sigma, Mo., USA).

Cell proliferation—Rat SMCs are seeded at density of 2×105 cells per petri dish (35 mm) and incubated with MEM supplemented with 10% FCS. Twenty-four hours later, the medium is changed with one containing 0.4% FCS to stop cell growth, and the cultures incubated for 72 h. After this time (time 0) the medium is replaced with one containing 10% FCS as mitogenic stimulus and various concentrations of the tested compounds. At time zero, just before the addition of drugs, three petri dishes are used for cell counting. Cell number is evaluated after 3 days of incubation by a Coulter Counter. On a separate group of Petri dishes immunoblot analyses of Ciclyn D and PCNA are performed. In another set of experiments, synchronization of SMC to the G0/G1 interphase of cell-cycle is accomplished by incubating logarithmically growing cultures (2.5×105 cells/plate) for 96-120 h in a medium containing 0.4% FCS. Quiescent cells are incubated for 20 h in a fresh medium with 10% FCS in the presence of the tested drugs. DNA synthesis is then estimated by nuclear incorporation of [3H] thymidine, incubated with cells (1 μCi/ml medium) for two hours. Radioactivity is measured with Aquasol scintillation cocktail (Packard, Groningen, NL).

Rabbit SMC are seeded at a density of 2×10⁵ cells per Petri dish (35 mm) and incubated with MEM supplemented with 10% FCS. Eighteen hours later the medium is changed with one containing 0.4% FCS to stop cell growth, and the cultures incubated for 48 h. After this time (time 0) the medium is replaced with one containing 10% FCS and various concentrations of compounds. At time zero, just before the addition of the drug, some Petri are used for cell counting. Cellular growth is evaluated by cell count after 1-7 days of incubation. Cell number is determined by Coulter Counter after trypsinization of the monolayers. Isoeffect curves are drawn as described.

Cyclin D and PCNA expression Cell monolayers are chilled, washed with cold phosphate-buffered saline (PBS), scraped in PBS containing a cocktail of protease inhibitors (Boehringer Mannheim) and centrifuged (2000 rpm, 10 min.). Cell pellets are then solubilized into 80 μl of sample buffer (3% SDS, 62.5 μM TRIS-HCl, pH=6.8 5% β-mercaptoethanol, 10% glycerol). 10-25 micrograms of protein are electrophoresed on 12% polyacrylamide or on 5-20% gradient gel for PCNA and cyclin D, respectively. Samples are electrophoretically transferred to Polivinylidene Fluoride membrane and incubated with anti cyclin D rabbit polyclonal antibody or with anti PCNA monoclonal antibody. Antibodies are detected with a donkey antirabbit and rabbit antimouse immunoglobin labelled with peroxidase conjugate. Peroxidase activity is revealed with ECL plus (Amersham).

Modifications of cyclin D and PCNA expression are evaluated by densitometric scanning of Western Blots and expressed as a mean percentage of the control conditions (10% FCS).

MMP expression and activity—MMP expression is evaluated by western blot analysis of the cell conditioned media using specific antibodies against human MMPs. MMP activity is measured by gelatin gel zymography.

Gelatin gel zymography—Proteins with proteinolytic activity are identified by electrophoresis on 7.5% polyacrilamide gels containing 10% SDS and gelatin (1 mg/ml) under non reducing conditions and without boiling. Then they are incubated overnight at 37° C. with gentle shaking in TRIS 50 mMol/l pH 7.5 containing NaCl 150 mM, CaCl₂ 10 mM, ZnCl₂ 1 □M, to activate the metalloproteinase ability to digest the substrate. At the end of the incubation, the gels are stained with Coomassie Blue. Clear zones against the blue background indicate the presence of proteinolytic activity.

Western Blot Analysis—Aliquots of the conditioned media (40 μl per lane) are run on 10% polyacrylamide gel containing SDS, under non-reducing conditions (Bellosta et al., 1998). The proteins are blotted to nitrocellulose membranes. (Bio-Rad Laboratories, Milan, Italy) and identified using a mouse monoclonal antibody anti-mouse MMP-9 (R & D).

Lipoproteins and lipoprotein deficient serum—Lipoproteins were prepared from the plasma of clinically healthy normolipidemic volunteers. LDL (d 1.019-1.063 g/ml) are isolated by sequential preparative ultracentrifugation and iodinated with ¹²⁵I. Radioactive LDL are used within three days from the preparation and sterilized by passage through a Millipore filter (0.22 μm pore size) immediately before incubation with the cells.

LDL binding uptake, and degradation—Confluent cells are preincubated for 48 h a 37° C. in a medium containing 10% human LPDS. After the 24 h pretreatment with lipoprotein-deficient medium to upregulate LDL receptor activity in the presence or absence of the tested compounds, each layer will received 1 ml fresh medium. ¹²⁵I-labelled LDL is added at the final concentrations of 7.5 μg/ml, and the cells incubated either at 37° C. for 4 h in lipoprotein-deficient medium or at 4° C. in medium A (supplemented with 10 mM Hepes buffer,) containing 10% lipoprotein-deficient serum. The cells are then placed on ice and washed three times with ice-cold phosphate-buffered saline, pH 7.4, containing 0.2% bovine serum albumin, and three times with ice-cold phosphate buffered saline.

Pre-chase treatment at 4° C. The cell layers were further washed by incubation at 4° C. for 30 min in medium A containing 10% lipoprotein-deficient serum. In certain experiments receptor-bound LDL are removed before the chase by exposure to sodium heparin. (10 mg/ml sodium heparin for 60 min at 4° C.). An aliquot of this medium is analysed for its content of ¹²⁵I (heparin releasable). After all pre-chase treatments, the cells are washed twice more with phosphate-buffered saline at 4° C.

For the chase at 37° C., cells receive 2 ml of lipoprotein-deficient medium. For the chase at 4° C., cells are exposed to ice-cold medium A containing 10% lipoprotein-deficient serum and kept on ice. After a 2 h chase period, the medium is removed and retained for analysis (see below). The cell layer is washed three times with phosphate-buffered saline and the cells dissolved by overnight incubation at 37° C. in 1 N NaOH. An aliquot of the solubilized layer is counted to determine the ¹²⁵I-radioactivity associated with cells, and an aliquot used for the estimation of cell protein according to the method of Lowry.

Medium analysis—0.3 ml 100% trichloroacetic acid will be added to 2 ml medium. After standing for 30 min on ice, the precipitate is collected by centrifugation at 1000×g for 30 min and the pellet counted for its content of ¹²⁵I-labelled trichloroacetic acid-precipitable material. A 1 ml aliquot of the acid supernatant is counted to determine the total trichloroacetic acid-soluble radioactivity and then is used for the determination of non-iodine trichloroactivity.

Synthesis of total sterols—The synthesis of cholesterol is determined by measuring the incorporation of radioactive acetate into cellular sterols. Cell monolayers, after incubation with [2-¹⁴C] acetate (1 μCi/ml) for 72 h, is washed with PBS and digested with 0.1 M NaOH. Aliquots are saponified at 60° C. for 1 h in alcoholic NaOH after the addition of [1,2(n)-³H]cholesterol as internal standard (0.04 μCi/sample). The nonsaponified material is extracted with low-boiling petroleum ether and counted for radioactivity. To evaluate the incorporation of labeled acetate into cellular sterols, these are separated from the nonsaponified fraction by thin-layer chromatography with use of petroleum ether (boiling point, 40-60° C.)/diethyl ether/acetic acid (70:30:1). Radioactivity is measured with Insta-Fluor scintillator cocktail (Packard, Milan, Italy).

Fatty acid synthesis—The aqueous phases from the petroleum ether extractions are pooled together, acidified with concentrated HCl, and extracted three times with petroleum ether. The pooled organic phases are then evaporated to dryness, resuspended in chloroform containing 100 μg of linoleic acid as carrier, and subjected to thin layer chromatography on Silica Gel G with a solvent system consisting of heptane/diethyl ether/acetic acid vapor and quantified as previously described for the measurement of cholesteryl 14C-esters. The data are expressed as the picomoles of [14C]acetate incorporated into 14C-fatty acids per mg of total cell protein.

Cholesterol esterification assay (ACAT activity): Cells are incubated with the tested drugs and AcLDL (50 μg/ml) as indicated. Cholesterol esterification is measured after addition of [1-¹⁴C]oleic acid (0.68 μCi/sample) complexed with bovine serum albumin during the last 2 h of incubation and subsequent determination of radioactivity associated with cellular cholesteryl esters.

At the end of incubation, cells are washed with PBS and lipids extracted with hexane/isopropanol (3:2). The extracted lipids are separated by TLC (isoctane/diethyl ether/acetic acid, 75:25:2, v/v/v). Cholesterol radioactivity in the spots is determined by liquid scintillation counting.

Sterol and cholesterol efflux—Cells are grown in 24-well plates until 80% confluent. Cells are labeled either by adding 30 μg/ml [3H]-Acetylated LDL for 24 hours or, to radiolabel cellular cholesterol, by adding 3 μCi/ml [1, 2-3H]cholesterol with 30 μg/ml Acetylated LDL for 24 hours. Cells are then incubated for 18 h with medium containing 0.2% BSA with or without HDL or apoAI.

Statistics—To ensure an unbiased result, morphometric data are collected in a blinded fashion. The specimens are ascribed to their respective treatment group after all numerical data were obtained. Data are expressed as mean ±SD. Differences between groups is evaluated by 1-way ANOVA followed by unpaired Student's t test. Statistical significance is assigned at the 95% confidence level (P<0.05).

It has surprisingly been found that, the combination of fluvastatin or a pharmaceutically acceptable salt thereof, and everolimus (40-0-(2-hydroxyethyl)-rapamycin) achieves greater therapeutic effect (a potentiation) in the prevention or the treatment of atherothrombosis.

More generally, It has also surprisingly been found that, a combination of an HMG-Co-A reductase inhibitor, especially fluvastatin or pitavastatin or a pharmaceutically acceptable salt thereof, and a mTOR inhibiting agent, e.g. rapamycin or a rapamycin derivative achieves greater therapeutic effect (a potentiation) in the prevention or the treatment of atherothrombosis.

The combinations according to the invention also surprisingly ameliorates symptoms and improves sides effect for example myotoxicity.

Greater efficacy can also be documented as a prolonged duration of action. The duration of action can be monitored as either the time to return to baseline prior to the next dose or as the area under the curve (AUC) and is expressed as the product of the change in blood pressure in millimeters of mercury (change in mmHg) and the duration of the effect (minutes, hours or days).

Further benefits are that lower doses of the individual drugs to be combined according to the present invention can be used to reduce the dosage, for example, that the dosages need not only often be smaller but are also applied less frequently, or can be used to diminish the incidence of side effects.

Preferred are low dose combination of HMG-Co-A reductase inhibitor and mTOR inhibiting agent. The combined administration of an HMG-Co-A reductase inhibitor especially fluvastatin or pitavastatin, or a pharmaceutically acceptable salt thereof and a mTOR inhibiting agent, e.g. rapamycin or a rapamycin results in a significant response in a greater percentage of treated patients, that is, a greater responder rate results, regardless of the underlying etiology of the condition. This is in accordance with the desires and requirements of the patients to be treated.

It can be shown that combination therapy with fluvastatin or pitavastatin agent and a mTOR inhibiting agent, e.g. rapamycin or a rapamycin derivative results in a more effective HMG-Co-A reductase inhibitors related conditions or diseases therapy such as hypercholesterolemia related conditions or diseases, mixed dyslipidemia related conditions or diseases, secondary prevention of cardiovascular event, atherosclerosis related conditions or diseases and mTOR inhibiting agent related conditions or diseases through improved efficacy as well as a greater responder rate.

It can further be shown that fluvastatin or pitavastatin and a mTOR inhibiting agent, e.g. rapamycin or a rapamycin derivative combination therapy proves to be beneficial in the reduction of side effect due to HMG-Co-A reductase inhibitors treatment, for example, reduction of toxicity.

Thus in the present description the terms “treatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition.

The Agents of the Invention, i.e. the HMG-Co-A reductase inhibitors or fibrates agent are preferably used in the form of pharmaceutical preparations that contain the relevant therapeutically effective amount of each active ingredient (either separately or in combination) optionally together with or in admixture with inorganic or organic, solid or liquid, pharmaceutically acceptable carriers which are suitable for administration. The Agents of the Invention may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping. The unit dose form may also be a fixed combination.

Preferably, the pharmaceutical compositions are adapted for oral or parenteral (especially oral) administration. Intravenous and oral, first and foremost oral, administration is considered to be of particular importance.

The pharmaceutical compositions according to the invention can be prepared in a manner known per se and are those suitable for enteral, such as oral, rectal, aerosol inhalation or nasal administration, and parenteral such as intravenous or subcutaneous administration, or compositions for transdermal administration (e.g. passive or iontophoretic) to mammals (warm-blooded animals), including man. Such compositions comprise a therapeutically effective amount of the pharmacologically active compound, alone or in combination with one or more pharmaceutically acceptable carriers, especially suitable for enteral or parenteral application. Typical oral formulations include tablets, capsules, syrups, elixirs and suspensions. Typical injectable formulations include solutions and suspensions.

Tablets may be either film coated or enteric coated according to methods known in the art. Preferred are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 85%, preferably about 1 to 70%, of the active ingredient.

The typical pharmaceutically acceptable carriers for use in the formulations described above are exemplified by: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, buffers, preservatives, antioxidants, lubricants, flavoring agents, and the like commonly used in pharmaceutical formulations.

Pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable formulations for topical application, e.g. to the skin and eyes, include aqueous solutions, suspensions, ointments, creams, gels or sprayable formulations, for example, for delivery by aerosol or the like.

For example, the pharmaceutical preparations consist of from about 0.1-90%, preferably of from about 1% to about 80%, of the active compounds. Pharmaceutical preparations for enteral or parenteral administration are, for example, in unit dose forms, such as coated tablets, tablets, capsules or suppositories and also ampoules. These are prepared in a manner which is known per se, for example using conventional mixing, granulation, coating, solubulizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances. The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Fluvastatin is supplied in the form of suitable dosage unit form, for example, a capsule or tablet, and comprising a therapeutically effective amount, e.g. from about 20 mg to about 80 mg, which may be applied to patients. The application of the active ingredient may occur up to three times a day, starting, e.g., with a daily dose of 20 mg or 40 mg per day, increasing via 40 mg daily and further to 80 mg daily. Preferably, fluvastatin is applied once a day or twice a day in patients with a dose of 80 mg or 40-milligram doses taken 2 times a day, respectively, each. Corresponding doses may be taken, for example, in the morning, at mid-day or in the evening.

Daily dosages for the fluvastatin will, of course, vary depending on a variety of factors, for example the compound chosen, the particular condition to be treated and the desired effect. In general, however, satisfactory results are achieved on administration of fluvastatin at daily dosage rates of the order of 20 to 80 mg/kg per day. A preferred daily dosage range is about from 20 to 40 mg per day mg as a single dose or in divided doses. Fluvastatin, may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 20 mg active ingredient, usually 40 mg, e.g. fluvastatin, together with one or more pharmaceutically acceptable diluents or carriers therefore.

Daily dosages for the mTOR inhibitor will, of course, vary depending on a variety of factors, for example the compound chosen, the particular condition to be treated and the desired effect. In general, however, satisfactory results are achieved on administration of the mTOR inhibitor at daily dosage rates of the order of ca. 0.01 to 5 mg/kg per day, particularly 0.5 to 5 mg/kg per day, as a single dose or in divided doses. A preferred daily dosage range is about from 0.1 to 30 mg as a single dose or in divided doses. The mTOR inhibitor, e.g. Compound A, may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.05 to 15 mg active ingredient, usually 0.25 to 10 mg, e.g. Compound A, together with one or more pharmaceutically acceptable diluents or carriers therefore.

Rapamycin or derivatives thereof are well tolerated at dosages required for use in accordance with the present invention. For example, the NTEL for Compound A in a 4-week toxicity study is 0.5 mg/kg/day in rats and 1.5 mg/kg/day in monkeys.

The person skilled in the pertinent art is fully enabled to select a relevant test model to prove the efficacy of a combination of the present invention in the hereinbefore and hereinafter indicated therapeutic indications.

The following examples illustrate the above-described invention; however, it is not intended to restrict the scope of this invention in any manner.

### EXAMPLES

### A) Examples of Fluvastatin Formulation

TABLE 1
Composition of one Lescol XL 80 mg film-coated tablet

| | Amount per |
|---|---|
| Ingredient | tablet (mg) |
| Tablet core | |
| Fluvastatin sodium^{1,2} | 84.24 |
| Cellulose microcrystalline/ | 111.27 |
| Microcrystalline cellulose fine | |
| powder | |
| Hypromellose/ | 97.50 |
| Hydroxypropyl methyl cellulose³ | |
| Hydroxypropyl cellulose⁴ | 16.25 |
| Potassium hydrogen carbonate/ | 8.42 |
| Potassium bicarbonate | |
| Povidone | 4.88 |
| Magnesium stearate | 2.44 |
| Water, purified⁵ | Q.S. |
| Core tablet weight | 325.00 |
| Coating | |
| Coating premix - Yellow (I)⁶ | 9.75 |
| Water, purified² | Q.S. |
| Total weight | 334.75 |

TABLE 2
Composition of one lescol
20 mg capsule

| | UNIT |
|---|---|
| | FORMULA |
| NAME OF INGREDIENTS | (mg) |
| Active substance | |
| Fluvastatin Sodium | 21.060 |
| Excipients | |
| Magnesium stearate | 1.050 |
| Sodium hydrogen carbonate | 2.000 |
| Talc | 9.430 |
| Cellulose microcrystalline, fine | 24.000 |
| powder | |
| Cellulose microcrystalline, | 33.220 |
| granular powder | |
| Maize starch, physically | 41.900 |
| modified | |
| Calcium Carbonate | 62.840 |

TABLE 3
Composition of one lescol
40 mg capsule

| | UNIT |
|---|---|
| | FORMULA |
| NAME OF INGREDIENTS | (mg) |
| Active substance | |
| Fluvastatin Sodium | 42.120 |
| Excipients | |
| Magnesium stearate | 2.100 |
| Sodium hydrogen carbonate | 4.000 |
| Talc | 18.860 |
| Cellulose microcrystalline, fine | 48.000 |
| powder | |
| Cellulose microcrystalline, | 66.440 |
| granular powder | |
| Maize starch, physically | 83.800 |
| modified | |
| Calcium Carbonate | 125.680 |

Examples of Pitavastatin formulation (Examples 1 to 7)

### Example 1

Core (percentage related to core weight):4.18 mg (5.225% wt) of drug substance, for example pitavastatin Ca-salts, 42.82 mg (53.525% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate.

HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 2

Core (percentage related to core weight): 8.36 mg (10.45% wt) of drug substance, for example pitavastatin Ca-salts, 38.64 mg (48.3% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate.

HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 3

Core (percentage related to core weight):16.72 mg (20.9% wt) of drug substance, for example pitavastatin Ca-salts, 30.28 mg (37.85% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate.

HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 4

Core (percentage related to core weight):3.135 mg (3.92% wt) of drug substance, for example pitavastatin Ca-salts, 43.865 mg (54.83% wt) of microcrystalline cellulose, 4 mg (5% wt) of HPMC (3 cps), 12.50 mg (15.625% wt) of HPMC (100 cps), 12.50 mg (15.625%) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 5

Core (percentage related to core weight): 6.27 mg (7.84% wt) of drug substance, for example pitavastatin Ca-salts, 40.73 mg (% 50.91 wt) of microcrystalline cellulose, 4 mg (5% wt) of HPMC (3 cps), 16.64 mg (20.8% wt) of HPMC (100 cps), 8.36 mg (10.45%) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 6

Core (percentage related to core weight):12.54 mg (15.675% wt) of drug substance, for example pitavastatin Ca-salts, 34.46 mg (43.075% wt) of microcrystalline cellulose, 4 mg (5% wt) of HPMC (3 cps), 18.75 mg (23.4375% wt) of HPMC (100 cps), 6.25 mg (7.8125% wt) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### Example 7

Core (percentage related to core weight):16.72 mg (20.9% wt) of drug substance, for example pitavastatin Ca-salts, 30.28 mg (37.85% wt) of microcrystalline cellulose, 4 mg (5% wt) of HPMC (3 cps), 20 mg (25% wt) of HPMC (100 cps), 5 mg (6.25% wt) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol

1: A pharmaceutical combination comprising an HMG-Co-A reductase inhibitor, especially fluvastatin or pitavastatin or a pharmaceutically acceptable salt thereof and a mTOR inhibiting agent.

2: Pharmaceutical combination according to claim 1 wherein the HMG-Co-A reductase inhibitor is fluvastatin or a pharmaceutically acceptable salt thereof.

3: Pharmaceutical combination according to claim 1 wherein the HMG-Co-A reductase inhibitor is pitavastatin or a pharmaceutically acceptable salt thereof.

4: Pharmaceutical combination according to claim 1, wherein the mTOR inhibiting agent is selected from rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578),40-0-(2-hydroxyethyl)-rapamycin, 32-deoxorapamycin and 16-pent-2-ynyloxy-32(S)-dihydro-rapamycin, or a pharmaceutically acceptable salt thereof, for simultaneous sequential or separate use.

5: Pharmaceutical combination according to claim 1 comprising pitavastatin or a pharmaceutically acceptable salt thereof and 40-0-(2-hydroxyethyl)-rapamycin for simultaneous, sequential or separate use.

6: Pharmaceutical combination according to claim 1 comprising fluvastatin or a pharmaceutically acceptable salt thereof and 40-0-(2-hydroxyethyl)-rapamycin for simultaneous, sequential or separate use.

7: Pharmaceutical combination according to claim 1 for the treatment or prevention of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases and mTOR inhibiting agent related conditions or diseases such as transplantation, Rheumatoid arthritis, Inflammatory Bowel Disease (IBD), chronic graft rejection, Restenosis following angioplasty, solid tumors, specially solid tumor invasiveness or symptoms associated with such tumor growth, xenotransplant rejection, graft versus host (GvH) disease, autoimmune diseases and inflammatory conditions (systemic lupus erythematosus (SLE), diabetes type I etc.), asthma, multidrug resistance, proliferative disorders (tumors, hyperproliferative skin disorders, e.g. psoriasis), uveitis, keratoconjuctivitis sicca, fungal infections, angiogenesis and inhibition of bone resorption.

8-14. (canceled)

15: A method of prevention or treatment of HMG-Co-A reductase inhibitors related conditions or diseases such as hypercholesterolemia related conditions or diseases, mixed dyslipidemia related conditions or diseases, secondary prevention of cardiovascular event, atherosclerosis related conditions or diseases and mTOR inhibiting agent related conditions or diseases comprising the administration of a combination according to claim 1 and a optionally pharmaceutically acceptable carrier to a mammal in need thereof.
